(19) United States
(12) Patent Application Publication
(10) Pub. No.: US 2017/0035279 A1
(43) Pub. Date: Feb. 9, 2017

(54) IMAGE PICKUP UNIT AND ELECTRONIC ENDOSCOPE INCLUDING THE IMAGE PICKUP UNIT

(71) Applicant: OLYMPUS CORPORATION, Tokyo (JP)

(72) Inventor: Toshiyuki FUJII, Tokyo (JP)

(73) Assignee: OLYMPUS CORPORATION, Tokyo (JP)

(21) Appl. No.: 15/297,239

(22) Filed: Oct. 19, 2016

Related U.S. Application Data
(63) Continuation of application No. PCT/JP2015/056906, filed on Mar. 10, 2015.

(30) Foreign Application Priority Data
Sep. 18, 2014 (JP) 2014-190334

(51) Int. Cl. A61B 1/05; G02B 23/24; H04N 5/225; A61B 1/00

(52) U.S. Cl. CPC A61B 1/051; A61B 1/00114; A61B 1/00124; A61B 1/053; G02B 23/2484; H04N 5/2253; H04N 2005/2255

(57) ABSTRACT

An image pickup unit includes: a circuit substrate electrically connected with an image pickup device that detects an image of an object, the circuit substrate including a land at least on a surface thereof; a connecting member having conductivity and fixed to the circuit substrate; a wiring connecting portion provided on the connecting member; a substrate connecting portion extended bent from a side portion of the wiring connecting portion, and electrically connected to a land; and a wiring configured to be connected to the wiring connecting portion and electrically connected to the land through the connecting member.

### CROSS REFERENCE TO RELATED APPLICATION

This application is a continuation application of PCT/JP2015/056906 filed on Mar. 10, 2015 and claims benefit of Japanese Application No. 2014-190334 filed in Japan on Sep. 18, 2014, the entire contents of which are incorporated herein by this reference.

### BACKGROUND OF THE INVENTION

1. Field of the Invention

The present invention relates to an image pickup unit disposed at a distal end portion of an insertion portion of an electronic endoscope.

2. Description of the Related Art

Electronic endoscopes have been used in medical fields or industrial fields, for example, and such electronic endoscopes are configured to be introduceable from outside to inside of a living body or structure to observe the inside of the living body or structure in which observation is difficult to perform, and include an image pickup unit for picking up an optical image.

An image pickup unit in an electronic endoscope includes an objective lens that forms an image of an object, and an image pickup device such as a CCD (charge coupled device) or a CMOS (complementary metal oxide semiconductor) sensor, in general, disposed on an image-forming surface of the objective lens.

As an image pickup unit provided in such an electronic endoscope, the image pickup unit disclosed in Japanese Patent Application Laid-Open Publication No. 2005-304876 is known, for example. In the conventional image pickup unit of the electronic endoscope, a technique is disclosed, according to which a solid-form relay member is vertically arranged at a connection terminal portion on a circuit substrate so as to enable a signal line to be easily connected to the connection terminal portion.

Incidentally, in recent electronic endoscopes, there is a desire for reduction in a diameter of an insertion portion, and in accordance with such a desire, size reduction of an image pickup unit is also required. In order to address the above-described circumstances, in such an image pickup unit, it is required to arrange a plurality of lands for electrically connecting a plurality of signal lines on a circuit substrate without wasting a space.

### SUMMARY OF THE INVENTION

An image pickup unit according to one aspect of the present invention includes: an image pickup device; a circuit substrate electrically connected with the image pickup device; a connecting member having conductivity and fixed to the circuit substrate; and a cable connected to the circuit substrate and the connecting member, wherein the circuit substrate includes: a bonding substrate portion electrically connected to a rear surface of the image pickup device and having an area substantially same as an area of the rear surface of the image pickup device; a laminated substrate portion extended rearward from a substantially center of the bonding substrate portion; a land for signal line arranged on at least a surface of the laminated substrate portion; and a land for ground arranged on at least the surface of the laminated substrate portion, wherein the connecting member includes: a wiring connecting portion provided superimposed on a proximal end surface of the laminated substrate portion in parallel with the proximal end surface; and a substrate connecting portion extended bent from a side portion of the wiring connecting portion, and electrically connected to the land for ground, and wherein the cable includes: a signal line configured to be connected to the land for signal line; and a shield bundle formed by bundling a ground line of the cable with a shield of the signal line, a distal end of the shield bundle being electrically connected to a plane of the wiring connecting portion which is superimposed on the proximal end surface.

An electronic endoscope according to one aspect of the present invention includes: an image pickup unit that includes: an image pickup device; a circuit substrate electrically connected with the image pickup device; a connecting member having conductivity and fixed to the circuit substrate; and a cable connected to the circuit substrate and the connecting member, wherein the circuit substrate includes: a bonding substrate portion electrically connected to a rear surface of the image pickup device and having an area substantially same as an area of the rear surface of the image pickup device; a laminated substrate portion extended rearward from a substantially center of the bonding substrate portion; a land for signal line arranged on at least a surface of the laminated substrate portion; and a land for ground arranged on at least the surface of the laminated substrate portion, wherein the connecting member includes: a wiring connecting portion provided superimposed on a proximal end surface of the laminated substrate portion in parallel with the proximal end surface; and a substrate connecting portion extended bent from a side portion of the wiring connecting portion, and electrically connected to the land for ground, and wherein the cable includes: a signal line configured to be connected to the land for signal line; and a shield bundle formed by bundling a ground line of the cable with a shield of the signal line, a distal end of the shield bundle being electrically connected to a plane of the wiring connecting portion which is superimposed on the proximal end surface; and an insertion portion that incorporates the image pickup unit in a distal end portion thereof.

### BRIEF DESCRIPTION OF THE DRAWINGS

FIG. 1 illustrates a configuration of an endoscope according to a first embodiment.

FIG. 2 is a plan view showing a configuration of an image pickup unit according to the first embodiment.

FIG. 3 is a side view showing the configuration of the image pickup unit according to the first embodiment.

FIG. 4 is a perspective view showing a configuration of a metal member according to the first embodiment.

FIG. 5 is an exploded perspective view showing the configuration of the image pickup unit according to the first embodiment.

FIG. 6 is a perspective view showing the configuration of the image pickup unit according to the first embodiment, which is viewed from one direction.

FIG. 7 is a perspective view showing the configuration of the image pickup unit according to the first embodiment, which is viewed from another direction.

FIG. 8 is a perspective view showing a configuration of an image pickup unit according to a first modified example of the first embodiment.

FIG. 9 is an exploded perspective view showing an image pickup unit according to a second modified example of the first embodiment.

FIG. 10 is a perspective view showing the configuration of the image pickup unit according to the second modified example of the first embodiment.

FIG. 11 is a perspective view showing a configuration of a metal member according to a third modified example of the first embodiment.

FIG. 12 is a perspective view showing a configuration of an image pickup unit according to the third modified example according to the first embodiment.

FIG. 13 is a perspective view showing a metal member according to a fourth modified example of the first embodiment.

FIG. 14 is a perspective view showing a configuration of an image pickup unit according to the fourth modified example of the first embodiment, which is viewed from one direction.

FIG. 15 is a perspective view showing the configuration of the image pickup unit according to the fourth modified example of the first embodiment, which is viewed from another direction.

FIG. 16 is a perspective view showing a configuration of a metal member according to a fifth modified example of the first embodiment.

FIG. 17 is a perspective view showing a configuration of an image pickup unit according to a fifth modified example of the first embodiment.

FIG. 18 is a perspective view showing a configuration of a metal member according to a sixth modified example of the first embodiment.

FIG. 19 is a perspective view showing a configuration of an image pickup unit according to the sixth modified example of the first embodiment.

FIG. 20 is a perspective view showing a configuration of a metal member according to a seventh modified example of the first embodiment.

FIG. 21 is a perspective view showing a configuration of a metal member according to an eighth modified example of the first embodiment.

### DETAILED DESCRIPTION OF THE PREFERRED EMBODIMENT

Hereinafter, a preferred embodiment of the present invention will be described with reference to the drawings. Note that, in each of the drawings used in the description below, a different scale size is used for each of the constituent elements in order to allow the constituent elements to be illustrated in a recognizable size in the drawings, and the present invention is not limited only to the number and shapes of the constituent elements, the ratio of the sizes of the constituent elements, and a relative positional relationship among the constituent elements shown in these drawings. In addition, in the description below, the up direction and the down direction viewed facing the paper surface are sometimes described as the upper portion and the lower portion of constituent elements.

First, an image pickup unit and an electronic endoscope according to one aspect of the present invention will be described below with reference to the drawings.

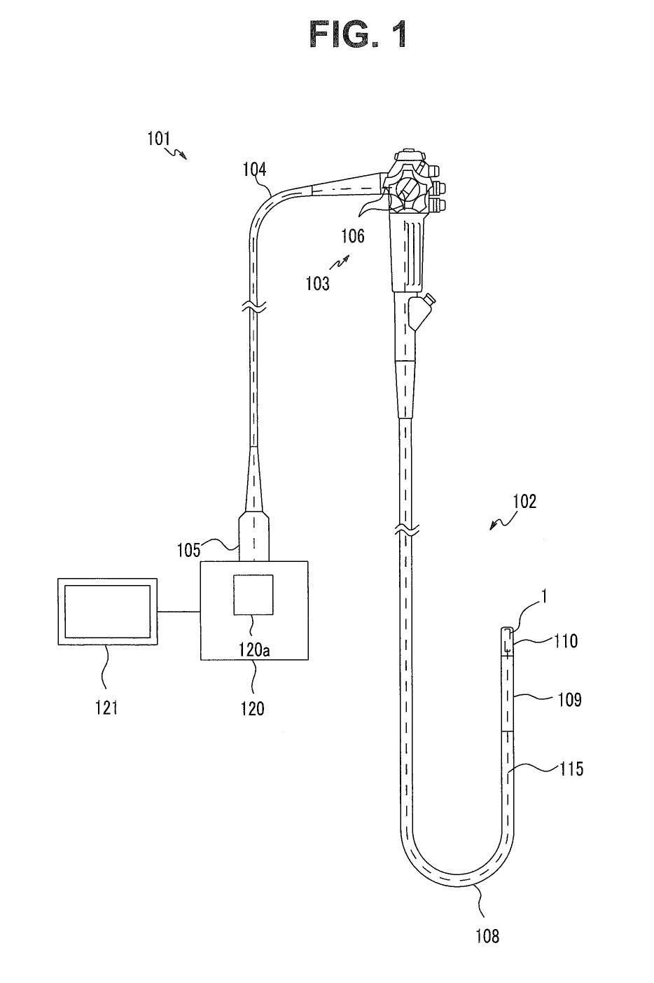

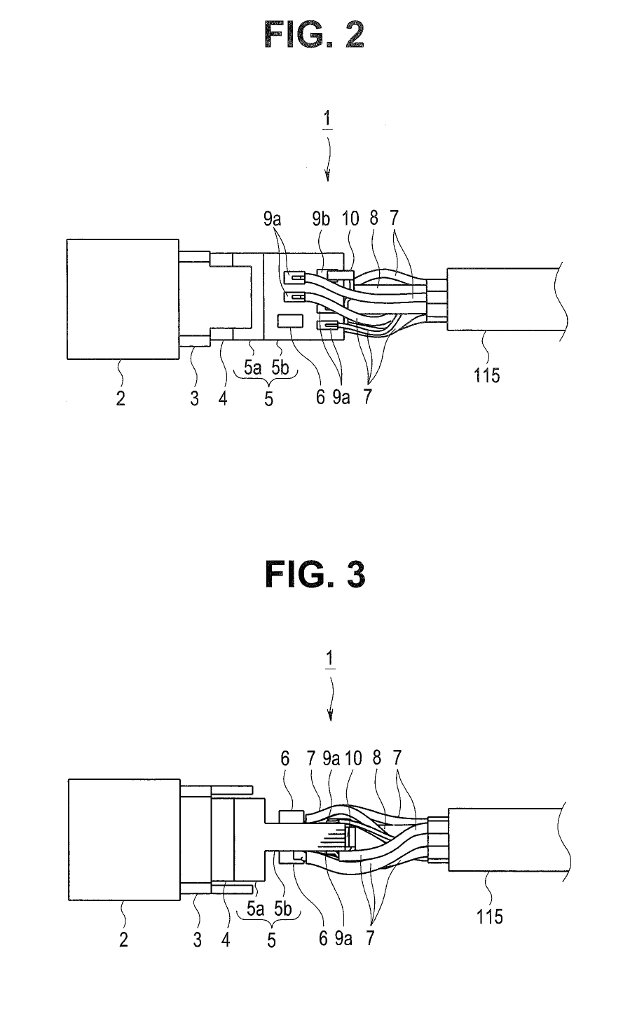

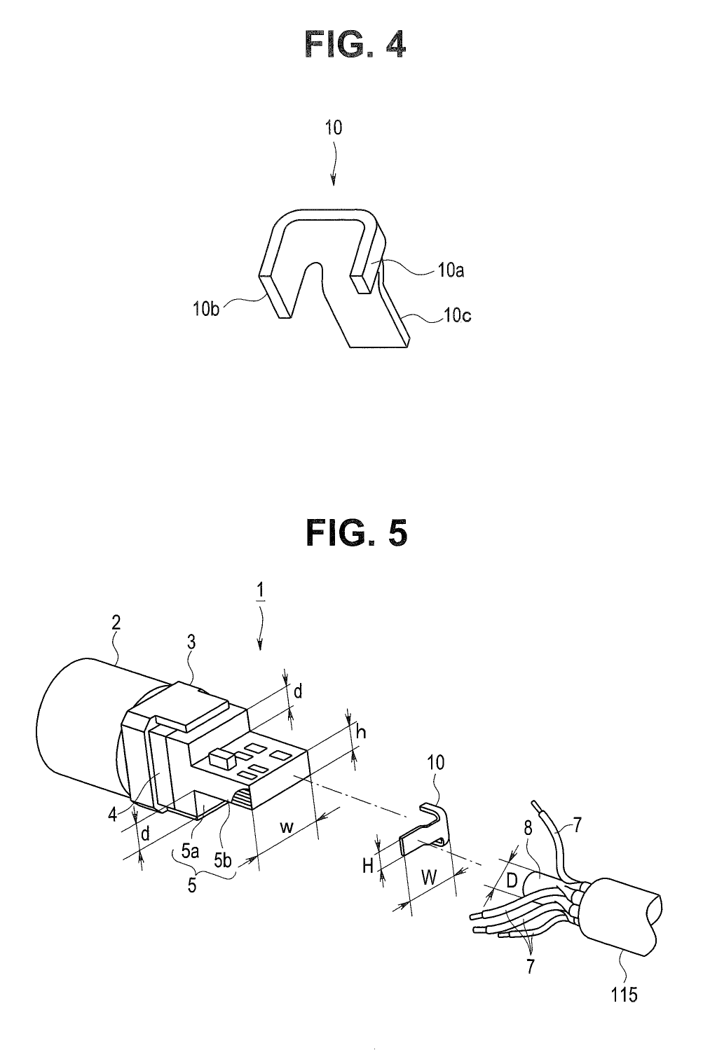

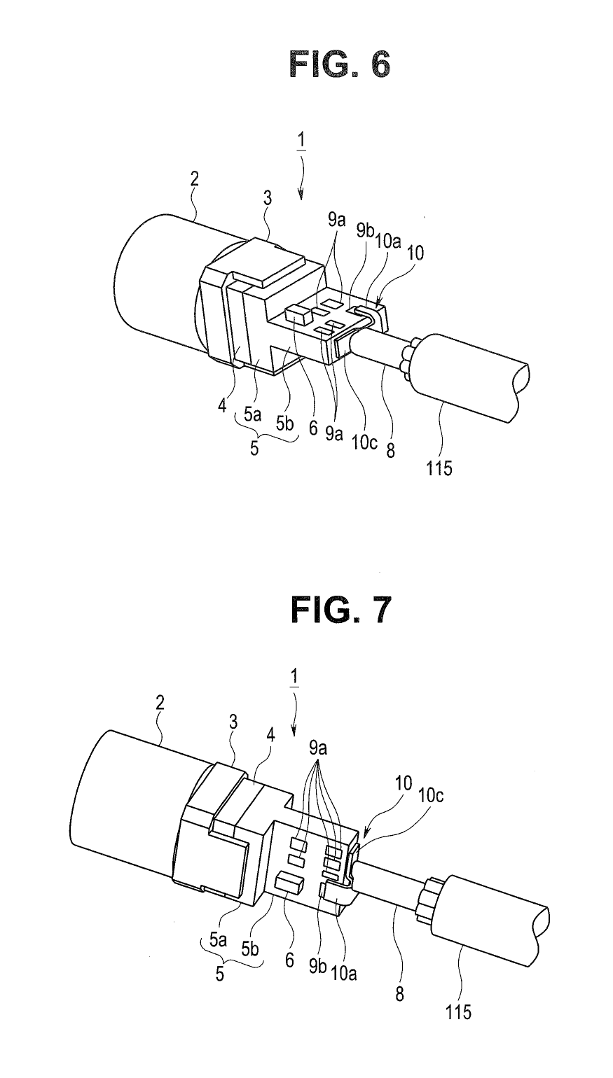

FIG. 1 illustrates a configuration of an endoscope, FIG. 2 is a plan view showing a configuration of an image pickup unit, FIG. 3 is a side view showing the configuration of the image pickup unit, FIG. 4 is a perspective view showing a configuration of a metal member, FIG. 5 is an exploded perspective view showing the configuration of the image pickup unit, FIG. 6 is a perspective view showing the configuration of the image pickup unit, which is viewed from one direction, and FIG. 7 is a perspective view showing the configuration of the image pickup unit, which is viewed from another direction.

First, with reference to FIG. 1, description will be made on one example of a configuration of an endoscope 101 including an image pickup unit 1 according to the present invention.

The endoscope 101 according to the present embodiment is configured to be introduceable into a subject such as a human body and optically pickup an image of a predetermined site to be observed in the subject.

Note that the subject into which the endoscope 101 is introduced is not limited to a human body, and may be another living body or an artificial object such as a machine or construction.

The endoscope 101 is mainly configured by an insertion portion 102 to be introduced into a subject, an operation portion 103 located on the proximal end with respect to the insertion portion 102, and a universal cord 104 extended from a side portion of the operation portion 103.

The insertion portion 102 is configured by a distal end portion 110 disposed at the distal end of the insertion portion, a bending portion 109 configured to be bendable and disposed on the proximal end side of the distal end portion 110, and a flexible tube portion 108 having flexibility provided on the proximal end side of the bending portion 109 and connected to the distal end side of the operation portion 103, which are provided in a linked manner.

Note that the endoscope 101 may be what is called a rigid endoscope which does not include a part having flexibility at the insertion portion 102.

The distal end portion 110 is provided with the image pickup unit 1, which will be detailed later. In addition, the operation portion 103 is provided with an angle operation knob 106 for operating bending of the bending portion 109.

The universal cord 104 includes at the proximal end portion thereof an endoscope connector 105 configured to be connected to an external apparatus 120. The external apparatus 120 to which the endoscope connector 105 is connected is connected to an image display section 121 such as a monitor through a cable.

In addition, the endoscope 101 includes a composite cable 115 inserted through the universal cord 104, the operation portion 103 and the insertion portion 102, and an optical fiber bundle (not shown) that transmits illumination light from a light source section provided in the external apparatus 120.

The composite cable 115 is configured to electrically connect the endoscope connector 105 and the image pickup unit 1. The endoscope connector 105 is connected to the external apparatus 120, and thereby the image pickup unit 1 is electrically connected to the external apparatus 120 through the composite cable 115.

Power supply from the external apparatus 120 to the image pickup unit 1 and communication between the external apparatus 120 and the image pickup unit 1 are performed through the composite cable 115.

The external apparatus 120 is provided with an image processing section. The image processing section generates a video signal based on an image pickup device output signal outputted from the image pickup unit 1, and outputs the generated video signal to the image display section 121. That is, in the present embodiment, the optical image (endoscopic image) picked up by the image pickup unit 1 is displayed on the image display section 121 as video.

Note that the endoscope 101 is not limited to be connected to the external apparatus 120 or the image display section 121. The endoscope 101 may be configured to include a part or all of the image processing section or the monitor, for example.

In addition, the optical fiber bundle is configured to transmit the light emitted from the light source section of the external apparatus 120 to an illumination window as an illumination light emitting portion of the distal end portion 110. Furthermore, the light source section may be disposed in the operation portion 103 or the distal end portion 110 of the endoscope 101.

Next, description will be made on the configuration of the image pickup unit 1 provided in the distal end portion 110. Note that, in the description below, there is a case where the direction from the image pickup unit 1 toward an object (left side in each of the drawings) is referred to as the distal end, the front side, or the object side, and the direction opposite to the above-described direction is referred to as the proximal end, the rear side, or the image side.

As shown in FIGS. 2 and 3, the image pickup unit 1 according to the present embodiment is configured by mainly including a lens holder 2, an image pickup device holding frame 3, an image pickup device 4, and a circuit substrate 5, in this order from the object side which is the front side.

In the lens holder 2, a plurality of objective lens groups as an objective optical system, not shown, are disposed. Note that the lens holder 2 is fitted to the image pickup device holding frame 3.

The image pickup device holding frame 3 holds an optical member such as a transparent glass plate, not shown, at the image side, and a transparent cover glass, not shown, for protecting the light-receiving portion of the image pickup device, is adhered to the image side of the optical member with an optical adhesive, and thereby the image pickup device holding frame 3 bonds and holds the image pickup device 4.

The image pickup device 4 is an extremely compact rectangular-shaped electronic component, a length of one side of which is about 2.0 mm in this embodiment. The image pickup device 4 is configured by aligning, on a planar light-receiving portion, a plurality of elements that output an electric signal corresponding to the incident light shown by the photographing optical axis O at a predetermined timing, and generally a type such as CCD (charge coupled device), CMOS (complementary metal oxide semiconductor) sensor, or various other types are applied to such an image pickup device 4, for example. The rear side, which is the proximal end side, of the image pickup device 4 is bonded to the circuit substrate 5.

The circuit substrate 5 has a multi-layer substrate which has a T-shaped cross section and which is configured by a laminated substrate whose base material is glass epoxy resin or ceramic. The circuit substrate 5 includes a bonding substrate portion 5a which is surface-bonded to the rear surface of the image pickup device 4 and has an area substantially same as that of the rear surface of the image pickup device 4, and a laminated substrate portion 5b as a planar block on which a plurality of electronic components 6 are mounted and which is formed so as to extend rearward from substantially the center of the bonding substrate portion 5a. That is, the circuit substrate 5 is positioned within the projected area of the outer frame of the image pickup device 4.

The laminated substrate portion 5b includes, on the front and rear surfaces thereof, a plurality of lands for signal lines 9a to which core lines of a plurality of signal lines 7 are connected with a brazing filler metal such as solder, and two lands for ground 9b to which a metal member 10 (see FIG. 4) as a shield connecting member to be described later is electrically connected with the brazing filler metal such as solder.

That is, on the circuit substrate 5, the plurality of signal lines 7 extended from the composite cable 115 and configured to transmit and receive an image pickup signal, a driving signal, and the like are electrically connected to the plurality of lands for signal lines 9a with the brazing filler metal such as solder. Note that coaxial cables are used for the signal lines 7 in the present embodiment.

In addition, on the circuit substrate 5, a shield bundle 8, as a shield wiring for ground, formed by bundling the braided shield of the plurality of signal lines 7 extended from the composite cable 115 and a ground line is electrically connected to the lands for ground 9b through the metal member 10. The shield bundle 8 is formed by bundling the braided shield and the ground line. Therefore, the diameter of the shield bundle 8 is larger than that of the plurality of signal lines 7.

Note that the level difference d between the front surface of the bonding substrate portion 5a and the front surface of the laminated substrate portion 5b of the circuit substrate 5 and the level difference d between the rear surface of the bonding substrate portion 5a and the rear surface of the laminated substrate portion 5b of the circuit substrate 5 are set to be smaller than the outer diameter (diameter) D of the shield bundle 8 (d<D). In addition, the height (thickness) h of the laminated substrate portion 5b is set to be equal to or larger than the outer diameter (diameter) D of the shield bundle 8 (h≧D) (see FIG. 5).

As shown in FIG. 4, the metal member 10 includes first and second terminal portions 10a, 10b, which are substrate connecting portions, formed by bending one substantially T-shaped sheet metal member made of stainless steel subjected to nickel plating so that the cross section of the sheet metal member has a U-shape, and a plate-like wiring connecting portion 10c which extends in the direction substantially orthogonal to the first and second terminal portions 10a, 10b and to which the distal end of the shield bundle 8 is electrically connected with solder or the like.

That is, the metal member 10 includes the first and second terminal portions 10a, 10b bent in the same one direction orthogonal to the plate surface, on one end portion side of the wiring connecting portion 10c.

The metal member 10 is formed such that the width W in the longitudinal direction (length of the wiring connecting portion 10c) is set to be smaller than the width w of the laminated substrate portion 5b (W<w), and the height H in the transverse direction (the width of the wiring connecting portion 10c) is set to be smaller than the height (thickness) h of the laminated substrate portion 5b (H<h). That is, also the metal member 10 is positioned within the projected area of the outer frame of the image pickup device 4.

The metal member 10 thus configured is, as shown in FIGS. 5 to 7 (note that illustration of the plurality of signal lines 7 is omitted in FIGS. 6 and 7), fixed such that the first and second terminal portions 10a, 10b sandwich the front and rear surfaces of the laminated substrate portion 5b therebetween from the proximal end surface side of the laminated substrate portion 5b of the circuit substrate 5.

That is, the separation distance between the first and second terminal portions 10a, 10b opposed to each other is set to be slightly smaller than the thickness of the laminated substrate portion 5b, and the metal member 10 is fixed to the laminated substrate portion 5b by the clamping force generated when the first and second terminal portions 10a, 10b sandwich the laminated substrate portion 5b therebetween.

Therefore, the metal member 10 is fixed such that the wiring connecting portion 10c has a predetermined angle, substantially right angle (˜90 degrees) with respect to the front and rear surfaces of the laminated substrate portion 5b.

Note that the metal member 10 is fixed to the laminated substrate portion 5b at the position where the first and second terminal portions 10a, 10b contact the lands for ground 9b disposed on the front and rear surfaces of the laminated substrate portion 5b so as to cover a part of the lands for ground 9b, to be electrically connected thereto.

Then, the respective lands for ground 9b to which the first and second terminal portions 10a, 10b are electrically connected so as to cover a part thereof are firmly connected with the first and second terminal portions 10a, 10b by flowing the brazing filler metal such as solder to the exposed part of the lands for ground 9b.

In addition, the wiring connecting portion 10c of the metal member 10 is electrically connected with the distal end of the shield bundle 8 using the brazing filler metal such as solder. Note that the shield bundle 8 is formed by bundling the braided shield of the plurality of signal lines 7 extended from the composite cable 115 and the ground line, and thereafter impregnated with the brazing filler metal such as solder in advance so as to prevent the bundling from coming apart.

Note that, as a procedure of fixing the shield bundle 8 to the circuit substrate 5, the metal member 10 is fixed to the laminated substrate portion 5b by electrically connecting the first and second terminal portions 10a, 10b to the respective lands for ground 9b, and thereafter the shield bundle 8 may be connected to the wiring connecting portion 10c, or the shield bundle 8 is connected to the wiring connecting portion 10c in advance and thereafter the metal member 10 may be fixed to the laminated substrate portion 5b such that the first and second terminal portions 10a, 10b are electrically connected to the respective lands for ground 9b.

Then, in the image pickup unit 1, the core lines of the plurality of signal lines 7 are connected to the plurality of lands for signal lines 9a provided on the laminated substrate portion 5b of the circuit substrate 5, with the brazing filler metal such as solder.

According to the image pickup unit 1 of the present embodiment which is configured as described above, even if the diameter of the shield bundle 8 becomes large by bundling the braided shield of the plurality of signal lines 7 extended from the composite cable 115 and the ground line, the shield bundle 8 can be electrically connected to the lands for ground 9b provided on the laminated substrate portion 5b of the circuit substrate 5, through the metal member 10.

Such a configuration eliminates the need for increasing the size of the lands for ground 9b disposed on the laminated substrate portion 5b on the circuit substrate 5 in accordance with the outer diameter of the shield bundle 8, which enables the lands for ground 9b to be effectively disposed on the limited space of the laminated substrate portion 5b.

That is, there is no need for providing a space for disposing large lands for ground 9b on the laminated substrate portion 5b, and the plurality of lands for signal lines 9a to which the core lines of the plurality of signal lines 7 are connected and the lands for ground 9b to which the first and second terminal portions 10a, 10b of the metal member 10 are connected can be efficiently arranged on the front and rear surfaces of the laminated substrate portion 5b of the circuit substrate 5, which enables the size of the image pickup unit 1 to be reduced.

In particular, the need for providing large lands for ground 9b on the laminated substrate portion 5b of the circuit substrate 5 is eliminated, which enables the length of the circuit substrate 5 to be reduced. As a result, the size of the image pickup unit 1 can be reduced.

Furthermore, even if the shield bundle 8 is in a hardened state as a result of impregnation with the brazing filler metal such as solder, the shield bundle 8 is extended substantially linearly from the distal end of the composite cable 115 and connected to the wiring connecting portion 10c of the metal member 10. Therefore, the shield bundle 8 is not required to be drawn out around the circuit substrate 5 to connect the shield bundle to the lands for ground 9b, as required in a conventional unit, and the size of the whole outer shape of the image pickup unit 1 can be reduced.

The shield bundle 8 is not required to be drawn out around the circuit substrate 5, which allows a room in the space for the plurality of signal lines 7 to be drawn out around the circuit substrate 5 in order to connect the plurality of signal lines 7 to the plurality of lands for signal lines 9a, and the size of the whole outer shape of the image pickup unit 1 is reduced.

Such configuration allows the plurality of signal lines 7 and the shield bundle 8 to be positioned within the projected area of the outer frame of the image pickup device 4 in the image pickup unit 1 (see FIGS. 2 and 3). As a result, the size of the image pickup unit 1 can be reduced.

In particular, the image pickup unit 1 satisfies the various dimensional relation among the circuit substrate 5, the shield bundle 8, and the metal member 10, as described with reference to FIG. 5, to thereby allow the plurality of signal lines 7 and the shield bundle 8 to be positioned within the projected area of the outer frame of the image pickup device 4. Such a configuration provides the effect of size reduction.

Furthermore, since there is no need for bending the thick shield bundle 8 to draw out the shield bundle in the image pickup unit 1, it is possible to reduce the distance from the proximal end surface of the laminated substrate portion 5b of the circuit substrate 5 to the end surface of the composite cable 115 from which the plurality of the signal lines 7 and the shield bundle 8 are extended. As a result, the size of the image pickup unit 1 can be reduced.

In addition, even if the diameter of the shield bundle 8 is large, the shield bundle 8 is not required to be drawn out around the circuit substrate 5, which enables the shield bundle 8 to be easily mounted and prevents the load from being applied when the shield bundle 8 is connected to the lands for ground 9b of the laminated substrate portion 5b.

With such a configuration, the size of the image pickup unit 1 according to the present embodiment can be reduced. Therefore, when the image pickup unit 1 is incorporated in the distal end portion 110 of the insertion portion 102 of the endoscope 101, also the size of the distal end portion 110 can be reduced, which contributes to reduction in the diameter size of the insertion portion 102.

Note that the number of the terminal portions included in the metal member 10 is not limited to two, i.e., the first and second terminal portions 10a, 10b, but two or more terminal portions which are brought into contact with and connected to the lands for ground 9b may be provided, as long as the number of the terminal portions is plural.

### Modified Examples

The image pickup unit 1 may be configured as shown in various modified examples to be described below. Note that it is needless to say that the respective configurations in the various modified examples to be described below can be combined.

### First Modified Example

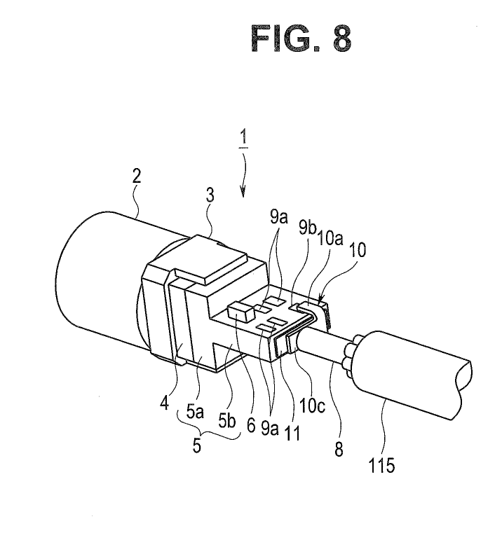

FIG. 8 is a perspective view showing a configuration of an image pickup unit according to the first modified example. Note that FIG. 8 omits the illustration of the plurality of signal lines 7.

As shown in FIG. 8, in the image pickup unit 1 according to the present modified example, an insulating tape 11 such as a polyimide tape is stuck on the proximal end surface of the laminated substrate portion 5b of the circuit substrate 5. The insulating tape 11 is thus stuck on the proximal end surface of the laminated substrate portion 5b, to thereby allow the insulating tape 11 to be interposed between the wiring connecting portion 10c and the laminated substrate portion 5b so as to be covered with the wiring connecting portion 10c of the metal member 10. As a result, insulation between the metal member 10 and the laminated substrate portion 5b can be ensured.

### Second Modified Example

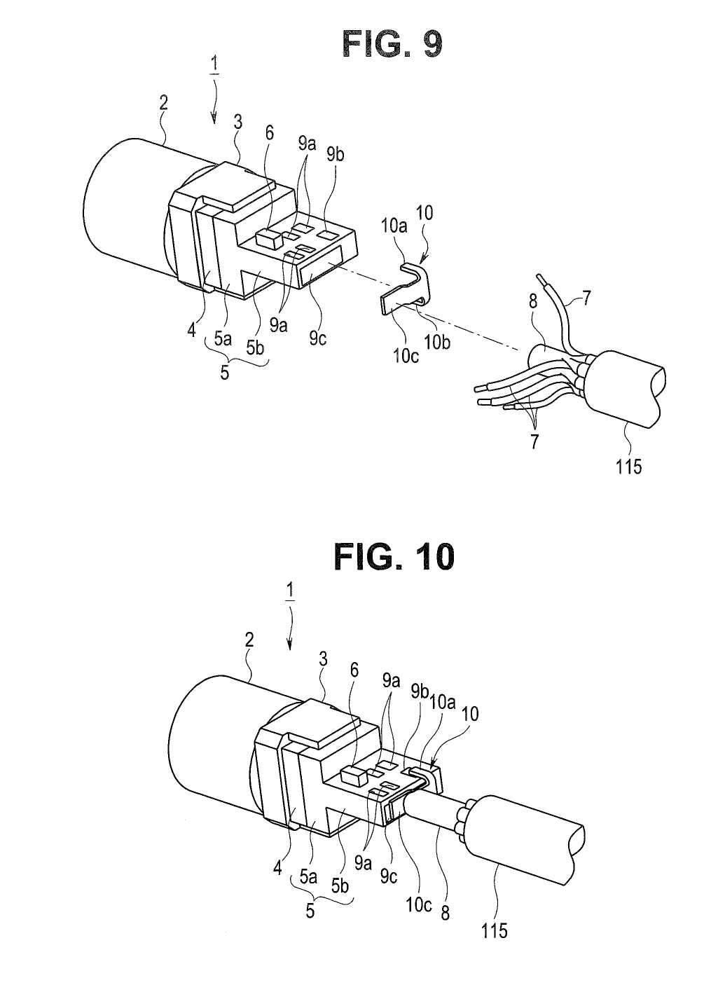

FIG. 9 is an exploded perspective view showing an image pickup unit according to the second modified example, and FIG. 10 is a perspective view showing the configuration of the image pickup unit. Note that also FIG. 10 omits the illustration of the plurality of signal lines 7.

The image pickup unit 1 according to the present modified example is provided with lands for ground 9c also on the proximal end surface of the laminated substrate portion 5b of the circuit substrate 5, as shown in FIG. 9. As shown in FIG. 10, the wiring connecting portion 10c of the metal member 10 is disposed on the lands for ground 9c so as to cover the lands for ground 9c, and the wiring connecting portion 10c is fixed with the brazing filler metal such as solder to be electrically connected to the lands for ground 9c.

The lands for ground 9c that are electrically connected with the metal member 10 are thus provided also on the proximal end surface of the laminated substrate portion 5b, which reduces resistance of connection between the circuit substrate 5 and the ground line through the metal member 10, to improve the conductivity, and improves the fixing strength of the metal member 10 to the laminated substrate portion 5b, compared with the metal member 10 that is electrically connected only with the lands for ground 9b provided on the front and rear surfaces, which are the land-formed surfaces, of the laminated substrate portion 5b.

### Third Modified Example

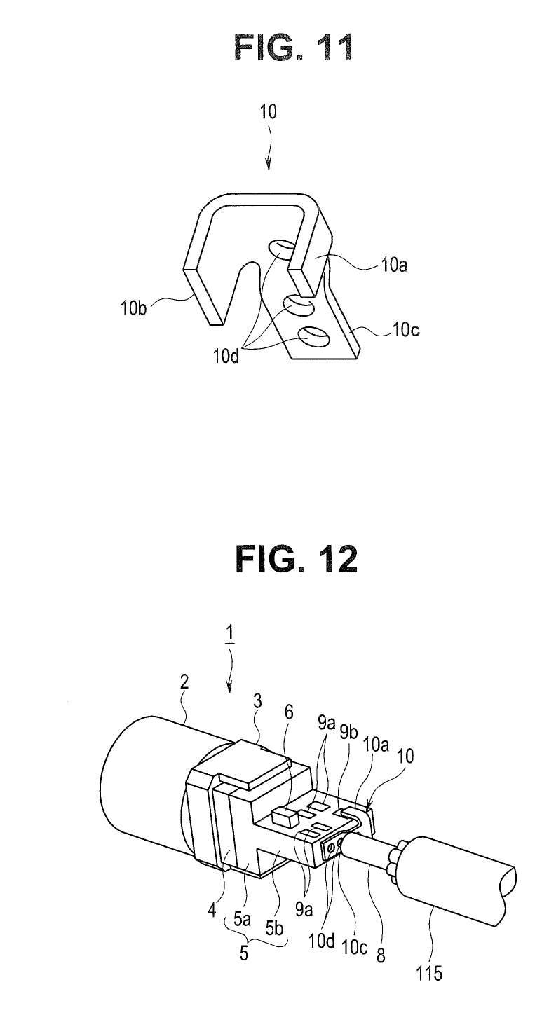

FIG. 11 is a perspective view showing a configuration of a metal member according to a third modified example, and FIG. 12 is a perspective view showing a configuration of an image pickup unit. Note that also FIG. 12 omits the illustration of the plurality of signal lines 7.

The image pickup unit 1 according to the present modified example includes a plurality of, three hole portions 10d in this modified example, which are drilled on the wiring connecting portion 10c of the metal member 10, as shown in FIG. 11. The metal member 10 is mounted on the proximal end surface of the laminated substrate portion 5b of the circuit substrate 5, as shown in FIG. 12.

The plurality of hole portions 10d are thus formed on the wiring connecting portion 10c of the metal member 10, to thereby allow the brazing filler metal such as solder to flow into the plurality of hole portions 10d at the time of connecting the shield bundle 8 and the wiring connecting portion 10c. As a result, the connecting workability between the shield bundle 8 and the wiring connecting portion 10c can be improved.

### Fourth Modified Example

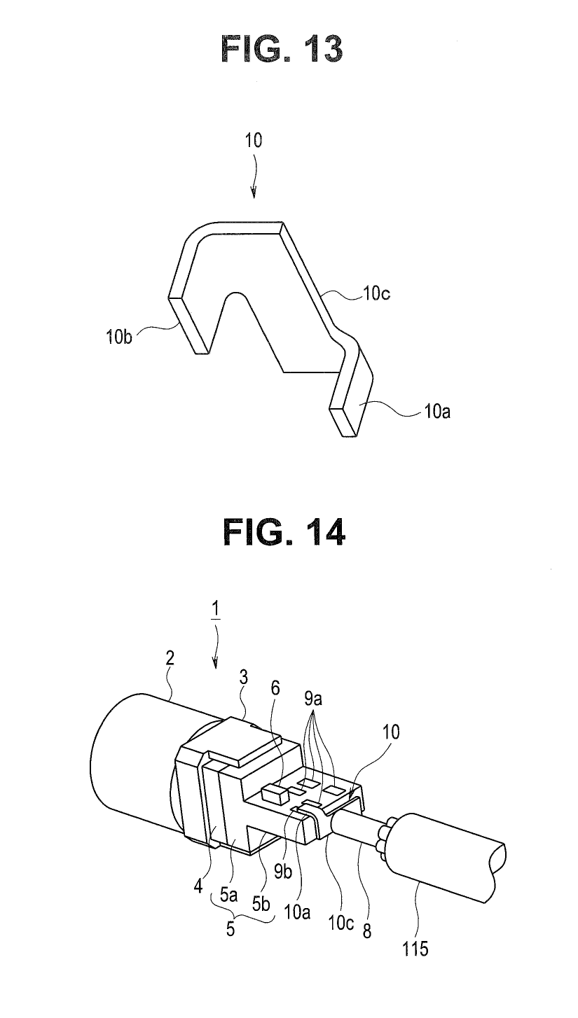

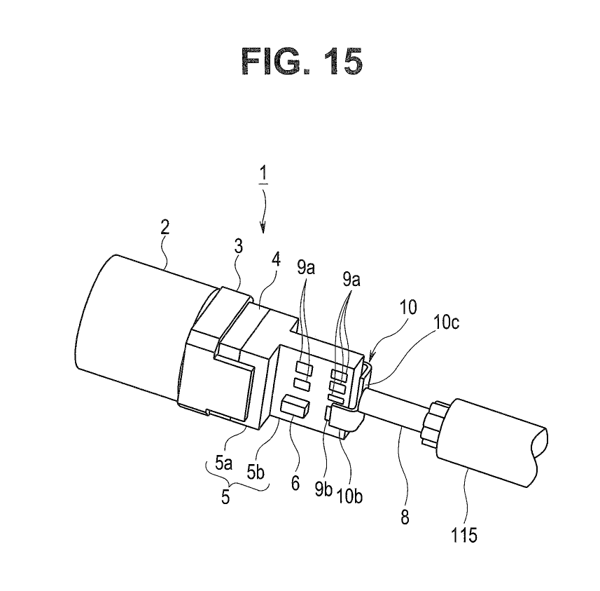

FIG. 13 is a perspective view showing a configuration of a metal member according to the fourth modified example, FIG. 14 is a perspective view showing a configuration of an image pickup unit, which is viewed from one direction, and FIG. 15 is a perspective view showing the configuration of the image pickup unit, which is viewed from another direction. Note that also FIGS. 14 and 15 omit the illustration of the plurality of signal lines 7.

In the image pickup unit 1 according to the present modified example, as shown in FIG. 13, the first and second terminal portions 10a, 10b of the metal member 10 are provided at end positions in the diagonal direction on the plate surface of the wiring connecting portion 10c, and the first and second terminal portions 10a, 10b are bent in the same one direction substantially orthogonal to the plate surface of the wiring connecting portion 10c.

The metal member 10 in the present modified example is fixed to the laminated substrate portion 5b at the position where the first and second terminal portions 10a, 10b contact the lands for ground 9b disposed on the front and rear surfaces of the laminated substrate portion 5b so as to cover a part of the lands for ground 9b, and electrically connected to the lands for ground 9b, as shown in FIGS. 14 and 15. Also in the present modified example, the metal member 10 is fixed to the laminated substrate portion 5b by the clamping force generated when the first and second terminal portions 10a, 10b sandwich the laminated substrate portion 5b therebetween.

Also in the present modified example, the lands for ground 9b to which the first and second terminal portions 10a, 10b are electrically connected so as to cover a part thereof are firmly connected with the first and second terminal portions 10a, 10b by flowing the brazing filler metal to the exposed part of the lands for ground 9b.

Note that the two lands for ground 9b in the present modified example are provided respectively on the front and rear surfaces of the laminated substrate portion 5b at the positions in the diagonal direction of the surface orthogonal to the front and rear surfaces of the laminated substrate portion 5b with which the first and second terminal portions 10a, 10b of the metal member 10 are brought into contact.

The configuration in which the first and second terminal portions 10a, 10b of the metal member 10 are thus provided at the end positions in the diagonal direction of the wiring connecting portion 10c is capable of improving the fixing strength of the metal member 10 with respect to the circuit substrate 5.

### Fifth Modified Example

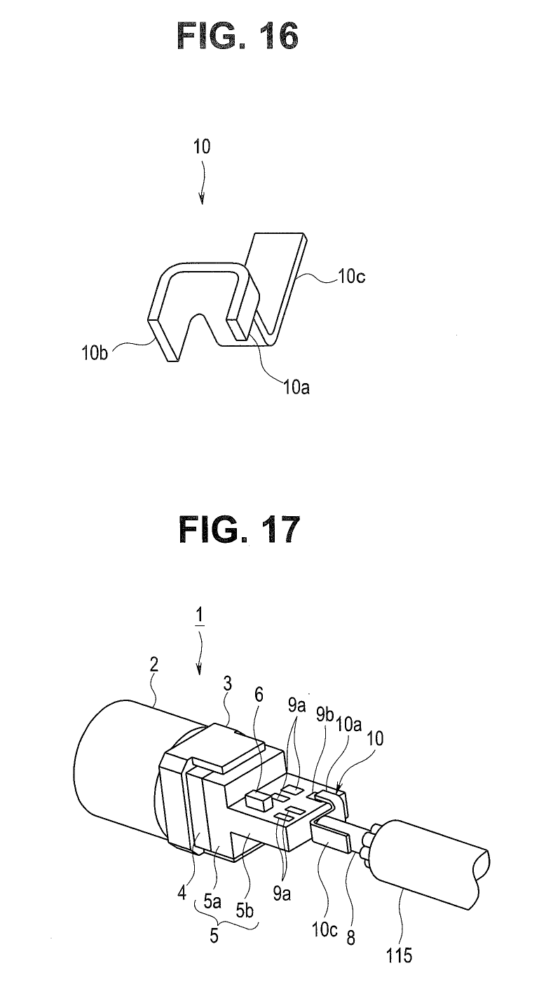

FIG. 16 is a perspective view showing a configuration of a metal member according to the fifth modified example, and FIG. 17 is a perspective view showing a configuration of an image pickup unit. Note that also FIG. 17 omits the illustration of the plurality of signal lines 7.

As shown in FIG. 16, in the image pickup unit 1 according to the present modified example, the wiring connecting portion 10c of the metal member 10 is bent from the halfway portion thereof at substantially right angle (90 degrees) in the direction opposite to the extended direction in which the first and second terminal portions 10a, 10b are bent.

According to the metal member 10 in the present modified example, as shown in FIG. 17, the wiring connecting portion 10c is fixed to the laminated substrate portion 5b so as to extend in the proximal end direction. Such a configuration enables the shield bundle 8 to be connected and fixed to the wiring connecting portion 10c with the brazing filler metal such as solder along the longitudinal axis direction.

The wiring connecting portion 10c of the metal member 10 which is fixed to the circuit substrate 5 is thus configured to extend in the proximal end direction of the circuit substrate 5. Such a configuration is capable of improving the connecting workability and connecting strength of the shield bundle 8 with respect to the wiring connecting portion 10c.

### Sixth Modified Example

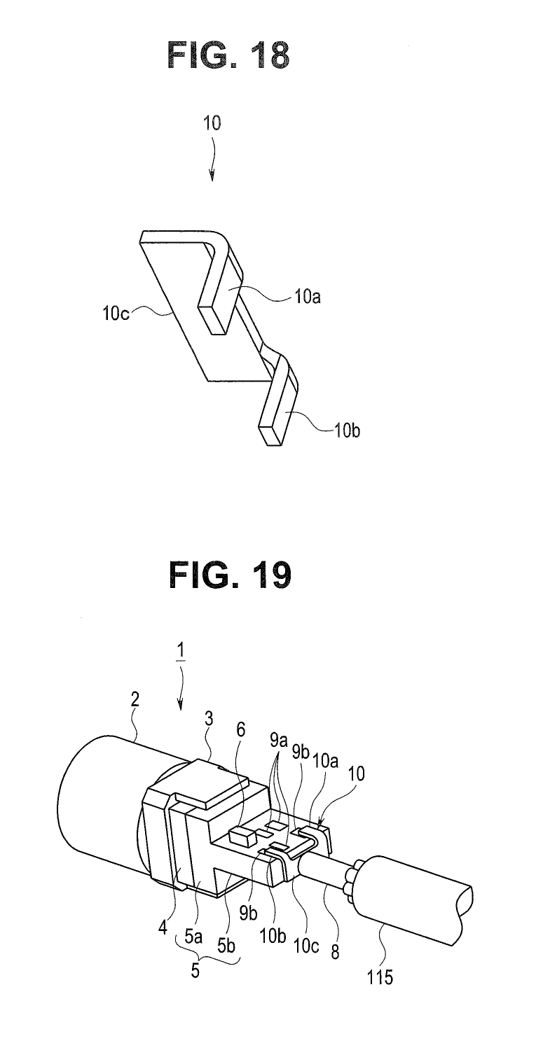

FIG. 18 is a perspective view showing a configuration of a metal member according to the sixth modified example, and FIG. 19 is a perspective view showing a configuration of an image pickup unit. Note that also FIG. 19 omits the illustration of the plurality of signal lines 7.

As shown in FIG. 18, in the image pickup unit 1 according to the present modified example, the first and second terminal portions 10a, 10b of the metal member 10 are provided respectively at positions of the both end portions of the same one side of the wiring connecting portion 10c, and the first and second terminal portions 10a, 10b are bent in the same one direction substantially orthogonal to the plate surface of the wiring connecting portion 10c.

As shown in FIG. 19, the metal member 10 according to the present modified example is fixed to the laminated substrate portion 5b at the position where the first and second terminal portions 10a, 10b contact the two lands for ground 9b disposed on one land-formed surface (one surface) of the laminated substrate portion 5b so as to cover a part of the lands for ground 9b, to be electrically connected thereto.

Also in the present modified example, the respective lands for ground 9b to which the first and second terminal portions 10a, 10b are electrically connected so as to cover a part thereof are firmly connected with the first and second terminal portions 10a, 10b by flowing the brazing filler metal to the exposed part of the lands for ground 9b.

Note that, in the present modified example, the two lands for ground 9b on the laminated substrate portion 5b are provided on the one land-formed surface (one surface) of the laminated substrate portion 5b with which the first and second terminal portions 10a, 10b of the metal member 10 are brought into contact.

Thus, the first and second terminal portions 10a, 10b of the metal member 10 are provided at the positions of the both end portions of the same one side of the wiring connecting portion 10c and the lands for ground 9b to which the first and second terminal portions 10a, 10b are electrically connected are provided on the one land-formed surface of the laminated substrate portion 5b. Such a configuration is capable of improving the degree of freedom of the layout of the electronic components 6 and the lands for signal line 9a that are provided on the front and rear surfaces of the laminated substrate portion 5b of the circuit substrate 5, in addition to the two lands for ground 9b.

### Seventh Modified Example

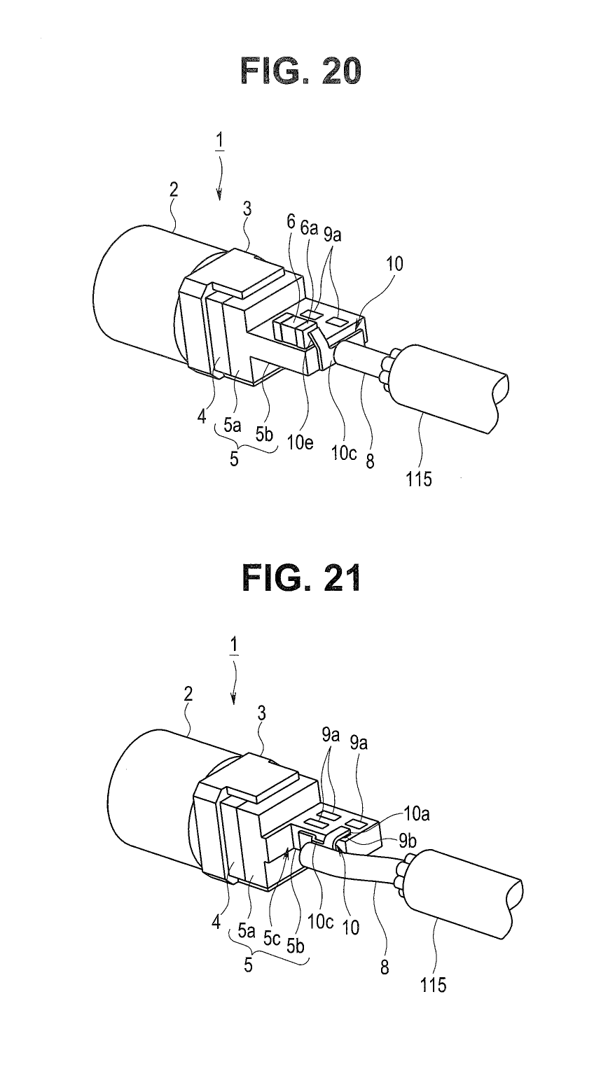

FIG. 20 is a perspective view showing a configuration of a metal member according to the seventh modified example. Note that also FIG. 20 omits the illustration of the plurality of signal lines 7.

As shown in FIG. 20, in the image pickup unit 1 according to the present modified example, a terminal portion 10e of the metal member 10 is provided so as to extend from one end portion of the side portion of the wiring connecting portion 10c, and the terminal portion 10e is connected to a terminal for ground 6a of an electronic component 6 which is a component mounted on the laminated substrate portion 5b of the circuit substrate 5.

Note that the terminal portion 10e is bent so as to be capable of contacting and being connected with the terminal for ground 6a of the electronic component 6. Then, the terminal portion 10e is connected and fixed to the terminal for ground 6a with brazing filler metal such as solder.

The metal member 10 is thus directly connected to the terminal for ground 6a of the electronic component 6, which eliminates the need for providing the lands for ground 9b on the laminated substrate portion 5b. As a result, the area of the land surface of the laminated substrate portion 5b can be reduced.

Note that the metal member 10 may include two or more terminal portions 10e, and configured to be connected also with the terminal for ground 6a of the electronic component 6 provided on the rear surface side of the laminated substrate portion 5b.

### Eighth Modified Example

FIG. 21 is a perspective view showing a configuration of a metal member according to the eighth modified example. Note that also FIG. 21 omits the illustration of the plurality of signal lines 7.

As shown in FIG. 21, the image pickup unit 1 according to the present modified example includes a cutout portion 5c formed by cutting out a part of the laminated substrate portion 5b of the circuit substrate 5 in a rectangular shape, and the metal member 10 is arranged on the side surface of the laminated substrate portion 5b, which is formed by the cutout portion 5c.

The wiring connecting portion 10c of the metal member 10 fixed to the side surface of the laminated substrate portion 5b has a surface along which the shield bundle 8 is arranged in the longitudinal axis direction. Therefore, the shield bundle 8 can be connected and fixed along the wiring connecting portion 10c with the brazing filler metal such as solder.

The wiring connecting portion 10c of the metal member 10 is thus fixed to the circuit substrate 5, with the shield bundle 8 being along the surface of the wiring connecting portion 10c, which enables the connecting workability and connecting strength of the shield bundle 8 to the wiring connecting portion 10c to be improved, and reduces the distance from the proximal end surface of the laminated substrate portion 5b to the end surface of the composite cable 115 from which the plurality of signal lines 7 and the shield bundle 8 are extended.

Note that, in the image pickup unit 1 according to the above-described embodiment and modified examples, what is called a vertical placing type image pickup device 4 is exemplified. However, the image pickup unit 1 can be applied to a configuration of what is called a horizontal placing type image pickup device that detects photographing light refracted by using a reflection member such as a prism, for example.

The inventions recited in the above-described embodiment and modified examples are not limited to the embodiment and modified examples, and various modifications can be practiced in the practical stage within a range without departing from the gist of the invention. Furthermore, the respective embodiments include inventions in various stages, and various inventions can be extracted by appropriately combining a plurality of disclosed constituent elements.

For example, even if some constituent elements are deleted from all the constituent elements shown in the embodiments and modified examples, the configuration from which some constituent elements are deleted can be extracted as the invention if the described problem can be solved and described effects can be obtained.

What is claimed is:

1. An image pickup unit comprising:
an image pickup device;
a circuit substrate electrically connected with the image pickup device;
a connecting member having conductivity and fixed to the circuit substrate; and
a cable connected to the circuit substrate and the connecting member,
wherein the circuit substrate includes:
a bonding substrate portion electrically connected to a rear surface of the image pickup device and having an area substantially same as an area of the rear surface of the image pickup device;
a laminated substrate portion extended rearward from a substantially center of the bonding substrate portion;
a land for signal line arranged on at least a surface of the laminated substrate portion; and
a land for ground arranged on at least the surface of the laminated substrate portion,
wherein the connecting member includes:
a wiring connecting portion provided superimposed on a proximal end surface of the laminated substrate portion in parallel with the proximal end surface; and
a substrate connecting portion extended bent from a side portion of the wiring connecting portion, and electrically connected to the land for ground, and
wherein the cable includes:
a signal line configured to be connected to the land for signal line; and
a shield bundle formed by bundling a ground line of the cable with a shield of the signal line, a distal end of the shield bundle being electrically connected to a plane of the wiring connecting portion which is superimposed on the proximal end surface.

2. The image pickup unit according to claim 1, wherein the substrate connecting portion includes a first terminal portion and a second terminal portion, and the first terminal portion and the second terminal portion sandwich a front surface and a rear surface of the laminated substrate portion therebetween from a proximal end surface side of the laminated substrate portion.

3. The image pickup unit according to claim 1, wherein an insulating sheet is provided between the wiring connecting portion and the circuit substrate.

4. The image pickup unit according to claim 1, wherein a plurality of hole portions are formed on the wiring connecting portion.

5. The image pickup unit according to claim 1, wherein the circuit substrate, the connecting member, and the shield bundle are disposed so as to be positioned within a projected area of an outer frame of the image pickup device.

6. The image pickup unit according to claim 1, wherein the connecting member is formed by one sheet of metal member.

7. An electronic endoscope comprising:
an image pickup unit comprising:
an image pickup device;
a circuit substrate electrically connected with the image pickup device;
a connecting member having conductivity and fixed to the circuit substrate; and
a cable connected to the circuit substrate and the connecting member,
wherein the circuit substrate includes:
a bonding substrate portion electrically connected to a rear surface of the image pickup device and having an area substantially same as an area of the rear surface of the image pickup device;
a laminated substrate portion extended rearward from a substantially center of the bonding substrate portion;
a land for signal line arranged on at least a surface of the laminated substrate portion; and
a land for ground arranged on at least the surface of the laminated substrate portion,
wherein the connecting member includes:
a wiring connecting portion provided superimposed on a proximal end surface of the laminated substrate portion in parallel with the proximal end surface; and
a substrate connecting portion extended bent from a side portion of the wiring connecting portion, and electrically connected to the land for ground, and
wherein the cable includes:
a signal line configured to be connected to the land for signal line; and
a shield bundle formed by bundling a ground line of the cable with a shield of the signal line, a distal end of the shield bundle being electrically connected to a plane of the wiring connecting portion which is superimposed on the proximal end surface; and
an insertion portion that incorporates the image pickup unit in a distal end portion thereof.
